(19) United States
(12) Patent Application Publication
(10) Pub. No.: US 2026/0311869 A1
(43) Pub. Date: Oct. 8, 2026

(54) METHOD, APPARATUS, AND COMPUTER-READABLE MEDIUM FOR GENERATING PREDICTIONS WITH A DIGITAL TWIN ARCHITECTURE

(71) Applicant: Intellisys D Corp, New York, NY (US)

(72) Inventor: Christian CORCINO, Rockaway beach, NY (US)

(21) Appl. No.: 19/097,680

(22) Filed: Apr. 1, 2025

Related U.S. Application Data
(60) Provisional application No. 63/572,652, filed on Apr. 1, 2024.
(60) Provisional application No. 63/635,162, filed on Apr. 17, 2024.

(51) Int. Cl. G16H 50/20; G16H 10/60; G16H 50/30

(52) U.S. Cl. CPC G16H 50/20; G16H 10/60; G16H 50/30

(57) ABSTRACT

An apparatus, computer-readable medium, and method for generating predictions with a digital twin architecture, including extracting data sets corresponding to a user from data sources, generating a digital twin model corresponding to the user based on the data sets, storing intelligent agents configured to interface with the digital twin model, each intelligent agent comprising a machine learning model corresponding to a defined feature set and being trained on historical data, the machine learning model being configured to generate a prediction, applying an intelligent agent to the digital twin model based on extracting data from the digital twin model corresponding to the defined feature set of the machine learning model and applying the machine learning model to the extracted data to generate a prediction, and transmitting an output based at least in part on the generated prediction.

### RELATED APPLICATION DATA

This application claims priority to U.S. Provisional Application No. 63/572,652, filed Apr. 1, 2024 and U.S. Provisional Application No. 63/635,162, filed Apr. 17, 2024, the disclosures of which are hereby incorporated by reference in their entirety.

### BACKGROUND

Current healthcare technologies individually contribute to healthcare by improving record-keeping, enabling remote consultations, assisting in diagnostic accuracy, and monitoring health metrics in real-time. Despite their utility, these systems operate in isolation, lacking the necessary integration to provide a cohesive view of a patient's health.

Additionally, current healthcare technologies face significant limitations that undermine their ability to deliver comprehensive, personalized, and proactive care. One critical issue is data fragmentation, as existing systems often operate in silos, lacking interoperability across diverse data sources such as electronic health records (EHRs), genomics, wearable devices, and medical imaging. These isolated data silos hinder seamless coordination among healthcare providers, making it difficult to form a unified view of a patient's health and compromising comprehensive care.

Moreover, many technologies provide limited personalization, offering generalized insights that fail to account for the unique characteristics of individual patients. This shortfall limits the ability to deliver tailored treatment strategies and actionable health recommendations. Compounding these issues is a widespread focus on reactive care, where technologies address symptoms only after they arise, rather than enabling prevention and early intervention through predictive analytics.

Operational inefficiency further exacerbates these challenges, as manual processes like claims validation and diagnostic reviews remain time-consuming, labor-intensive, and prone to human error. These inefficiencies reduce the capacity of healthcare providers to focus on delivering quality care. Additionally, scalability challenges persist, as many existing systems are designed for narrow applications and lack the flexibility to adapt to diverse healthcare contexts or the evolving health conditions of patients. Existing systems also fail to incorporate real-time contextual factors such as lifestyle or environmental changes, leaving patients without dynamic, adaptive solutions.

Accordingly, improvements are needed in systems for data integration, predictive analytics, machine learning, data monitoring, and personalized healthcare recommendations.

### BRIEF DESCRIPTION OF THE DRAWINGS

FIG. 1 illustrates a flowchart for generating predictions with a digital twin architecture according to an exemplary embodiment.

FIG. 2 illustrates a system chart showing different types of data sources that can be used to extract the plurality of data sets according to an exemplary embodiment.

FIG. 3 illustrates a flowchart for generating a digital twin according to an exemplary embodiment.

FIG. 4 illustrates a system diagram of a digital twin platform and coupled components according to an exemplary embodiment.

FIG. 5 illustrates a flowchart for applying an intelligent agent in the one or more intelligent agents to the digital twin model based at least in part on extracting data from the digital twin model corresponding to the defined feature set of the machine learning model and applying the machine learning model to the extracted data to generate a prediction when the one or more intelligent agents include a risk prediction intelligent agent according to an exemplary embodiment.

FIG. 6 illustrates a flowchart for applying an intelligent agent in the one or more intelligent agents to the digital twin model based at least in part on extracting data from the digital twin model corresponding to the defined feature set of the machine learning model and applying the machine learning model to the extracted data to generate a prediction when the one or more intelligent agents include a diagnostic intelligent agent according to an exemplary embodiment.

FIG. 7 illustrates an example vector space used to predict diagnosis according to an exemplary embodiment.

FIG. 8 illustrates a flowchart for applying an intelligent agent in the one or more intelligent agents to the digital twin model based at least in part on extracting data from the digital twin model corresponding to the defined feature set of the machine learning model and applying the machine learning model to the extracted data to generate a prediction when the one or more intelligent agents include an efficacy intelligent agent according to an exemplary embodiment.

FIG. 9 illustrates a specialized computing environment for generating predictions with a digital twin architecture according to an exemplary embodiment.

### DETAILED DESCRIPTION

While methods, apparatuses, and computer-readable media are described herein by way of examples and embodiments, those skilled in the art recognize that methods, apparatuses, and computer-readable media for generating predictions with a digital twin architecture are not limited to the embodiments or drawings described. It should be understood that the drawings and description are not intended to be limited to the particular form disclosed. Rather, the intention is to cover all modifications, equivalents, and alternatives falling within the spirit and scope of the appended claims. Any headings used herein are for organizational purposes only and are not meant to limit the scope of the description or the claims. As used herein, the word “can” is used in a permissive sense (i.e., meaning having the potential to) rather than the mandatory sense (i.e., meaning must). Similarly, the words “include,” “including,” and “includes” mean including, but not limited to.

The present system and platform (referred to herein as “Hospitium” and “Hospitium.AI”) represents a paradigm shift in healthcare by addressing critical challenges such as data fragmentation, reactive care approaches, and a lack of holistic integration in existing healthcare systems. The platform integrates digital twin technology with advanced artificial intelligence to deliver adaptive, patient-centered solutions that bridge these gaps. Through interoperable systems, Hospitium seamlessly integrates data from multiple sources, including electronic health records (EHRs), wearable devices, and genomic information, creating comprehensive and dynamic digital twins for each patient.

These digital twins serve as the foundation for personalized care, providing precise, individualized health insights, tailored treatments, and wellness plans that adapt to the evolving conditions of each patient. Hospitium.AI also focuses on proactive health management by employing continuous monitoring and predictive analytics, enabling early detection of risks, prevention of complications, and improved health outcomes.

The platform's innovative design enhances operational efficiency by automating claims validation, diagnostic assistance, and workflow optimization, significantly reducing administrative burdens and minimizing human errors. Its scalable AI architecture ensures versatility, enabling applications across individual, clinical, and population health levels, making it adaptable to diverse healthcare settings.

Unlike traditional reactive systems, Hospitium shifts the focus towards prevention and early intervention, resolving the issue of fragmented data and fostering seamless coordination among healthcare providers. By combining real-time medical and behavioral data with domain-specific AI systems, Hospitium delivers actionable, tailored insights that empower both patients and care teams. This holistic integration not only improves decision-making and collaboration but also optimizes healthcare outcomes, transforming the process into a proactive, efficient, and personalized paradigm.

Hospitium.AI represents a groundbreaking advancement in healthcare technology, introducing a comprehensive platform that integrates dynamic digital twins, intelligent agents, and advanced interoperability to revolutionize patient care. At its core, Hospitium.AI creates real-time, high-fidelity digital twins-virtual replicas of patients generated from diverse data sources such as electronic health records (EHRs), wearable devices, genomic profiles, and behavioral inputs. These digital twins enable continuous monitoring, precise simulation of evolving health conditions, predictive diagnostics, and tailored therapeutic planning.

A key innovation of Hospitium.AI is the incorporation of Intelligent Agents (IA Agents), including Gallenus, an AI-powered virtual doctor (discussed further below). These agents interact seamlessly with digital twins to analyze complex datasets, simulate clinical scenarios, and provide actionable insights. Using advanced machine learning models, AI Agents dynamically adapt recommendations to patients' changing health conditions, ensuring a highly personalized approach to care.

The platform's multi-level functionality operates across individual, clinical, and population health domains, adapting to various healthcare scenarios—from personal wellness management to optimizing resources for healthcare systems. Its automation capabilities streamline critical but time-intensive processes, such as claims validation and diagnostic reviews, reducing administrative burdens and minimizing human errors.

Hospitium.AI's scalable and interoperable design bridges traditionally siloed healthcare domains, integrating specialties such as primary care, oncology, and mental health into a unified system. This facilitates seamless collaboration among providers and delivers a comprehensive view of each patient's health profile. Additionally, the platform supports “what-if” simulations, enabling clinicians to explore hypothetical scenarios and optimize clinical decisions without real-world risks.

With its holistic integration, predictive analytics, and proactive monitoring, Hospitium.AI transforms healthcare into a personalized, preventive, and adaptive system, addressing critical gaps in current technologies and setting a new standard for precision medicine.

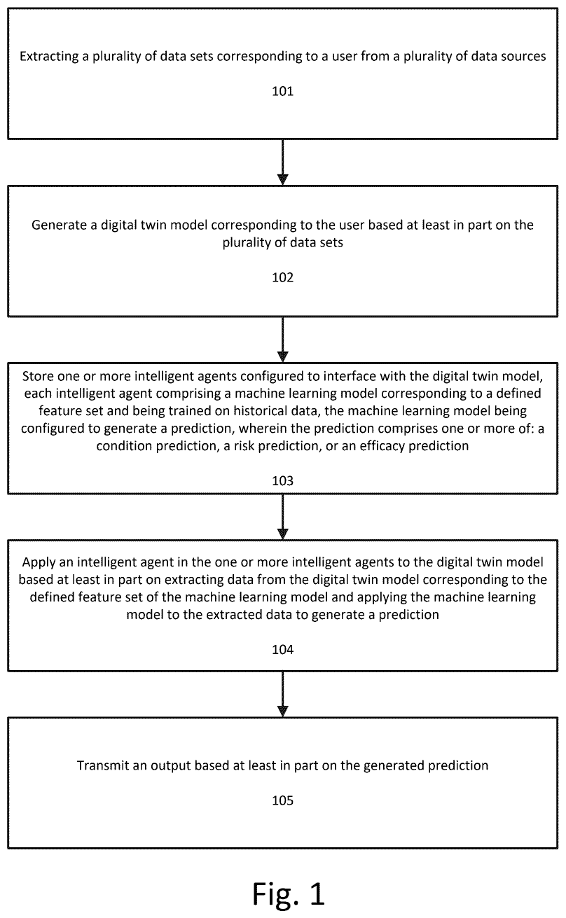

FIG. 1 illustrates a flowchart for generating predictions with a digital twin architecture according to an exemplary embodiment. Each of the steps shown in FIG. 1 can be performed by one or more computing devices of a controller of the Hospitium system.

At step 101 a plurality of data sets corresponding to a user are extracted from a plurality of data sources. The user can be a patient in the system or a future patient of the system. The (Fast Healthcare Interoperability Resources) FHIR-based data extraction process retrieves structured information from multiple medical sources. The demographic section includes patient identification, age, gender, weight, height, and provider details. The medical history component compiles past diagnoses, ICD-10/ICD-11 (International Classification of Diseases) codes, chronic conditions, and comorbidities, while the immunization record ensures accurate tracking of vaccine history and booster schedules. The system also collects allergy data, identifying medication, food, and environmental allergens that may impact treatment plans. Medication and treatment history logs current and past prescriptions, adherence patterns, and pharmacogenomic insights to personalize drug therapy. The system also integrates laboratory and diagnostic test results, including blood tests, biomarker panels, and imaging reports using DICOM (Digital Imaging and Communications in Medicine) standards for medical imaging compatibility.

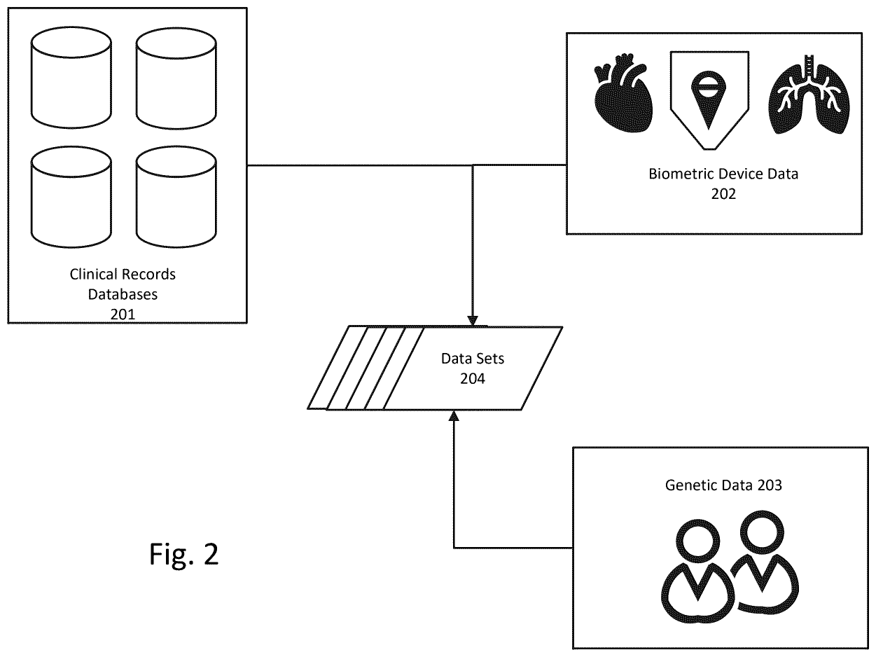

FIG. 2 illustrates a system chart showing different types of data sources that can be used to extract the plurality of data sets according to an exemplary embodiment. Multiple different data sources in each category of data sources can be used to extract the plurality of data sets.

As shown in FIG. 2, the plurality of data sources can include clinical records databases 201. The system can ingest structured and unstructured health data from electronic health records (EHRs), laboratory systems, medical imaging databases, clinical reports, and Electronic Medical Records (EMRs) through FHIR (Fast Healthcare Interoperability Resources) and HL7 (Health Level Seven) protocols. The extracted data can include:

Demographic Information—Patient age, sex, ethnicity, weight, height, lifestyle factors (e.g., smoking, alcohol use).

Medical History & Diagnoses—Past and current conditions, ICD-10/ICD-11 classifications, chronic conditions, prior hospitalizations, surgeries, allergies.

Immunizations & Allergy Records—Vaccine history, medication, environmental allergens, immunization status, and future schedule reminders.

Vital Signs & Clinical Observations: Blood pressure, temperature, oxygen saturation (SpO2), heart rate.

Laboratory & Imaging Results—Blood panels, biomarkers, radiology reports (DICOM), blood glucose, lipid profiles, liver function tests, inflammatory markers.

Radiology & Imaging Data: X-rays, MRIs, CT scans, ultrasounds (processed via AI for pattern recognition).

Medication & Treatment History—Prescription records, dosages, adherence patterns.

Doctor's Notes & Diagnoses: NLP-powered processing of physician notes to extract insights.

Mental Health & Behavioral Data—Psychiatric assessments, validated surveys (PHQ-9, GAD-7).

Patient-Reported Outcomes & Lifestyle Factors—Social determinants of health, physical activity, diet.

Clinical and health data can be ingested via FHIR APIs (Application Programming Interfaces), NLP (natural language processing) for free-text extraction, and cloud-based secure storage. As discussed below, the AI agents of the present system can analyze historical health trends and responses to treatments to improve predictions.

The plurality of data sources can also include biometric device data or databases storing biometric data 202. The system can integratee continuous, real-time biometric data from wearable devices and (Internet of Things) IoT-connected sensors to ensure dynamic update a digital twin.

The extracted real-time metrics and/or biometric data can include:

### Cardiovascular Data:

Heart rate (smartwatches, electrocardiogram (ECG) monitors).
Electrocardiogram (ECG) signals (medical-grade ECG wearables).
Blood pressure (smart BP cuffs).
Respiratory & Oxygenation Data:
Respiratory rate (chest sensors, smart rings).
Blood oxygen saturation (pulse oximeters, wearables).

### Metabolic & Blood Glucose Data:

Continuous glucose monitoring (CGMs like Dexcom, Abbott FreeStyle Libre).
Insulin monitoring (smart insulin pens).

### Neurological & Cognitive Data:

EEG (electroencephalography) sensors for brain activity analysis.
Sleep cycle tracking (smart rings, sleep trackers).

### Physical Activity & Mobility:

Step count, gait analysis, sedentary time (accelerometers in wearables).
Posture tracking and rehabilitation monitoring (motion sensors).

### Temperature & Hydration:

Skin temperature (smart thermometers, infrared sensors).
Hydration status tracking (bioimpedance wearables).

The biometric and real-time data can be collected via Bluetooth, WiFi, or cloud APIs and processed using real-time Artificial Intelligence (AI) algorithms. The AI algorithms detect anomalies, sends alerts, and adjusts health insights dynamically.

Hospitium.AI can extract real-time metrics from wearable devices and physiological sensors. The system incorporate continuous health monitoring, enabling real-time physiological data to be processed and analyzed to improve diagnostic accuracy, treatment optimization, and disease progression modeling within the digital twin system. These physiological parameters are sourced from smartwatches, fitness trackers, continuous glucose monitors (CGMs), smart rings, biofeedback headbands, and other IoT-enabled health sensors.

Hospitium.AI's architecture is structured to integrate and analyze real-time biometric data using a modular IoT-enabled system. Wearable devices will transmit health data via Bluetooth, Wi-Fi, or cellular networks, connecting through secure APIs with platforms like Apple Health, Google Fit, and Fitbit API. By incorporating real-time health metrics, the intelligent agents (such as Gallenus) provide personalized treatment recommendations, optimize medication plans, and improve diagnostic precision. Additionally, physicians have access to real-time patient monitoring dashboards, enabling them to make data-driven clinical decisions while reducing reliance on retrospective data.

Additionally, the plurality of data sources can include genetic or genomic data 203 extracted from genomic databases or systems. Hospitium.AI integrates genomic and multi-omics data for precision medicine and risk assessment.

The extracted genomic data can include:
Whole Exome Sequencing (WES) & Whole Genome Sequencing (WGS): Used for detecting mutations and predispositions to genetic disorders.
Polygenic Risk Scores (PRS): The system calculate the genetic likelihood of diseases (e.g., cardiovascular disease, diabetes, cancer).
Pharmacogenomics: The system predicts individual drug responses based on genetic markers.
Epigenetic Markers: The system analyzes lifestyle impacts on genetic expression for preventive healthcare.

The genomic data can be uploaded securely from sequencing providers (via APIs or bulk data import). The system then matches genetic predispositions with clinical data and lifestyle factors for personalized recommendations.

Hospitium.AI integrates genomic data analysis into a digital twin (discussed further below), allowing for predictive risk assessment, pharmacogenomics, and disease modeling based on an individual's genetic profile. The system extracts key genomic markers from whole genome sequencing (WGS), whole exome sequencing (WES), and clinical genetic testing services. The genomic data includes single nucleotide polymorphisms (SNPs) associated with disease susceptibility, pharmacogenomic data to optimize drug metabolism and dosing, polygenic risk scores (PRS) for complex diseases, and epigenetic markers reflecting environmental and lifestyle influences on gene expression.

To ensure secure and efficient genomic data processing, the system is designed with a modular, AI-driven pipeline. First, genomic data is acquired from clinical genetic laboratories, research databases, and direct-to-consumer platforms, following FHIR-based genomic standards for seamless EHR integration. Next, machine learning algorithms interpret genetic variations, classifying them based on clinical relevance and disease risk. Predictive analytics models then assess an individual's susceptibility to chronic conditions such as cardiovascular disease, diabetes, and neurodegenerative disorders. This data is then integrated into a digital twin, allowing AI-driven health simulations that factor in genetic predispositions, lifestyle behaviors, and medical history. Based on this information, intelligent agents that form part of the system (such as Gallenus) provide personalized treatment recommendations, particularly in pharmacogenomics, ensuring optimized drug therapy.

As new genetic discoveries emerge, the present system continuously refines risk assessments, updating genomic risk profiles and treatment strategies accordingly. Clinicians receive AI-generated reports summarizing genetic risk factors, recommended interventions, and preventive care strategies. The system implements an FHIR-compliant genomic data processing system that enhances AI-driven predictive modeling for genetic risk assessment and ensures compliance with international genomic data privacy standards such as HIPAA, GDPR, and GINA. The genomic data is a key component of the digital twin, and allows for precision medicine, personalized treatment planning, and proactive health management.

Returning to FIG. 1, at step 102 a digital twin model (also referred to herein as the “digital twin”) corresponding to the user is generated based at least in part on the plurality of data sets. The digital twin is a high-fidelity virtual representation of a patient, built using structured and unstructured data extracted from electronic health records (EHRs), medical reports, and predictive modeling algorithms. As discussed above, the system collects comprehensive patient information, including immunizations, allergies, treatments, medical history, family history, surveys, and medical reports, ensuring that it reflects an up-to-date and precise model of the patient's health status. FHIR-compliant data structures allow for seamless integration and interoperability with healthcare infrastructures, standardizing patient data for advanced analysis and simulation. The digital twin is a real-time AI-driven virtual representation of a patient, continuously updated with clinical, physiological, and genomic data. The dynamic updating enables predictive diagnostics, treatment simulations, and real-time patient monitoring through intelligent agents (such as Gallenus).

The process of generating a digital twin includes multiple stages, such as data acquisition, processing, and continuous updates to maintain an accurate and dynamic representation of the patient's health.

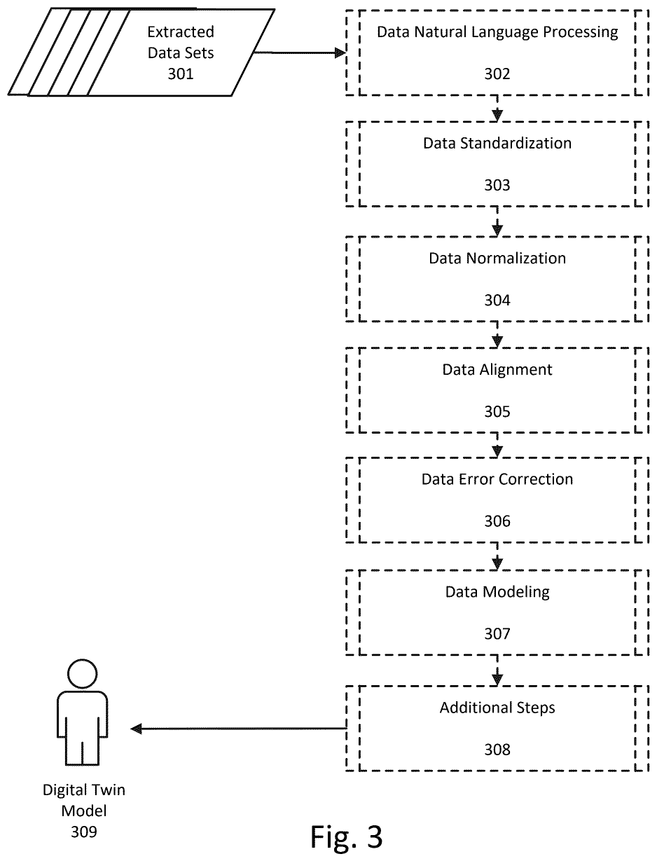

FIG. 3 illustrates a flowchart for generating a digital twin according to an exemplary embodiment. The process of generating the twin transforms the extracted data sets 301 into the digital twin model 309 and can include one or more of steps 302-308, shown in FIG. 3. The process can implement different steps for different types of data. For example, genomic data sets can be processed differently than health records.

At step 302 natural language processing is performed on unstructured data in the plurality of data sets, such as to extract relevant data, concepts, or semantic meaning.

At step 303 data formats in the plurality of data sets are standardized, such as to be in a consistent format.

At step 304 data in the plurality of data sets is normalized, such as to account for different ranges of values or metrics.

At step 305 data in the plurality of data sets is aligned, so that data from different data sources can be compared and combined.

At step 306 error correction of data in the plurality of data sets is performed, such as to fix errors such as missing data, improperly formatted data, or other data integrity issues.

At step 307 one or more relationships between data in the plurality of data sets can be modeled. The modeling can be performed with predictive and AI algorithms to identify trends or correlations, estimate future predicted values, or generate models for changes to the data based on inputs (such as disease progression, symptoms, treatments, etc.).

The process can also include one or more additional steps 308 not specifically enumerated above. Some of the above steps and the process for generating the digital twin are described in additional detail below.

Once data is extracted, it can be converted into FHIR-compliant resources to ensure uniformity across different healthcare systems. The system can map unstructured clinical notes using Natural Language Processing (NLP) to extract relevant medical terms and standardize them into FHIR formats. The system can further validates and normalizes numerical and categorical data, such as lab values, vital signs, and medication dosages, ensuring compatibility with AI-driven modeling. Additionally, the system can align diagnostic codes and treatment history with global medical classifications (ICD, SNOMED CT, LOINC) for consistency.

The system utilizes AI-driven data fusion strategies to generate the digital twin. The data normalization and standardization steps utilizes AI systems to clean, format, and align data from different sources (EHR, wearables, genomics). The data modeling steps can include utilizing machine learning algorithms to map relationships between genetic risk, lifestyle factors, and medical history. The AI systems can also dynamically link clinical data with real-time physiological signals for continuous monitoring.

Hospitium.AI further ensures that the digital twin remains dynamically updated by integrating real-time clinical inputs from EHR updates, lab test results, and patient interactions. The system synchronizes with healthcare systems via APIs, automatically updating patient profiles whenever new data is recorded. The system additionally monitors trends in treatment response and refines predictions based on updated health information. The system also generates automated alerts for clinicians, flagging changes in vital parameters, medication interactions, or early signs of disease progression. The digital twin continuously evolves as new health data is ingested. This enables the system detects early warning signs and sends alerts to doctors via intelligent agents, such as Gallenus.

The digital twin can be accessed through a user interface, such a clinician dashboard. The digital twin and dashboard can be used to provide AI-driven insights for personalized treatment recommendations based on real-time data, predictive outcome modeling, allowing clinicians to compare intervention strategies, and/or risk assessment reports, highlighting key health trends and early warning signs. As explained further below, the digital twin can be used as a simulator to simulate different health conditions in the patient (e.g., heart failure risk, diabetes progression). The digital twin can also be used to simulate different treatment strategies virtually before applying them to the real patient.

Returning to FIG. 1, at step 103 one or more intelligent agents configured to interface with the digital twin model are stored, each intelligent agent comprising a machine learning model corresponding to a defined feature set and being trained on historical data, the machine learning model being configured to generate a prediction, wherein the prediction comprises one or more of: a condition prediction, a risk prediction, or an efficacy prediction.

The one or more intelligent agents can include a plurality of intelligent agents. The plurality of intelligent agents can include one or more of:
risk prediction intelligent agent configured to predict future health risks and/or conditions based at least in part on the digital twin model;
a diagnostic intelligent agent configured to predict a diagnosis based at least in part on the digital twin model and/or symptom information that is provided by the user/patient;
an efficacy intelligent agent configured to simulate and predict the efficacy of a one or more courses of treatment, types of treatment, and/or drug regiments on a patient based at least in part on the digital twin model;
a well-being optimization intelligent agent configured to predict actions, activities, or other recommendations to optimize well-being based at least in part on the digital twin model; and/or
a mental health support intelligent agent configured to predict mental health risks and therapy recommendations based at least in part on the digital twin model.

The risk prediction intelligent agent, diagnostic intelligent agent, and efficacy intelligent agent can all be implemented as a single intelligent agent—a “virtual doctor” agent that is referred to in this document as “Gallenus.” It is understood that Gallenus can be implemented as multiple distinct intelligent agents as well. For example, Gallenus can refer to the combination of the risk prediction intelligent agent, diagnostic intelligent agent, and efficacy intelligent agent. Additionally, the well-being optimization intelligent agent is referred to herein as “Séneca” and the mental health support intelligent agent is referred to herein as “Flourish.” Additional details regarding the various intelligent agents are provided below.

As discussed above, in addition to Gallenus, Hospitium.AI incorporates two other key Intelligent Agents (AI Agents): Séneca and Flourish, each designed to address distinct but complementary aspects of well-being. Séneca is an AI-powered mental health assistant that supports healthcare professionals by providing personalized psychological assessments, early detection of mental health conditions, and therapy recommendations. It is specifically designed to assist clinicians in Cognitive Behavioral Therapy (CBT), offering structured interventions based on key CBT principles such as cognitive restructuring, behavioral activation, and emotion regulation. Séneca functions as a decision-support tool, helping professionals detect early warning signs of mental health conditions and tailor interventions accordingly. By analyzing patient data, Séneca enhances clinical decision-making, ensuring that mental health professionals can provide more targeted and effective support.

Flourish is an intelligent agent dedicated to well-being and lifestyle optimization, using Martin Seligman's PERMA-H model as its foundation. This model focuses on Positive Emotions, Engagement, Relationships, Meaning, Accomplishment, and Health, ensuring a comprehensive approach to well-being. Flourish helps individuals develop and maintain healthy habits by integrating data from nutrition, sleep, physical activity, and social interactions to provide personalized AI-driven coaching. It tracks daily wellness patterns, detects imbalances, and offers recommendations to optimize routines, enhance social connections, and support long-term personal growth. Unlike Séneca, which is designed to support mental health professionals, Flourish is user-centric, empowering individuals to take an active role in improving their physical, emotional, and social well-being. Together, these agents create a seamless AI-driven ecosystem, ensuring that mental health support, clinical guidance, and lifestyle optimization are fully integrated within Hospitium.AI.

Together, these Intelligent Agents complement Gallenus by ensuring that mental health, lifestyle habits, and clinical care are seamlessly integrated within Hospitium.AI, delivering a comprehensive, AI-driven health ecosystem that promotes long-term well-being.

Séneca and Flourish, the intelligent agents within Hospitium.AI, are built using a modular AI architecture that integrates machine learning models, natural language processing (NLP), real-time data processing, and behavioral science frameworks. This design allows them to provide adaptive learning, real-time decision-making, and personalized recommendations, ensuring a dynamic and data-driven approach to mental health support (Séneca) and lifestyle optimization (Flourish).

Séneca's architecture consists of several layers: a data ingestion layer that collects structured and unstructured data from clinical notes, psychometric assessments, and wearable inputs, an AI decision engine that applies NLP models to analyze psychological text inputs and detect early warning signs of emotional distress, and a CBT-based intervention module that generates structured therapy recommendations. Additionally, Séneca features a clinician dashboard that presents patient mental health trends, therapy progress tracking, and AI-generated clinical reports, ensuring compliance with HIPAA and GDPR regulations for secure mental health data handling.

Flourish's architecture is centered around real-time health monitoring, behavioral analytics, and AI-driven habit formation. The data acquisition layer integrates wearables and health tracking platforms to collect biometric and behavioral data, while its behavioral analytics engine applies reinforcement learning models to analyze user engagement and predict habit formation success rates. Additionally, Flourish provides AI-powered coaching, social well-being analysis, and gamification techniques to encourage sustained behavioral change. Through a dynamic user dashboard, individuals receive personalized wellness insights, adaptive health recommendations, and interactive goal tracking.

The specific functions of each of the intelligent agents, along with examples, are provided in greater detail below with respect to the application of intelligent agents to the digital twin model.

At step 104 of FIG. 1 an intelligent agent in the one or more intelligent agents is applied to the digital twin model based at least in part on extracting data from the digital twin model corresponding to the defined feature set of the machine learning model and applying the machine learning model to the extracted data to generate a prediction.

The intelligent agent that is applied to the digital twin model will depend on the specific task that the platform is currently performing. For example, if the platform is engaged in a triage process and attempting to diagnose a condition, then the diagnostic intelligent agent can be applied to the digital twin model. In another example, if the platform is building a new digital twin model for the first time (i.e., for a new patient), then the risk prediction intelligent agent can be applied to predict any future health risks for the patient based on the generated digital twin model. In another example, if the platform is being used by a clinician to evaluate the efficacy of different treatments, then the efficacy intelligent agent can be applied to the digital twin model.

The process of applying the intelligent agent to the digital twin model includes extracting data from the digital twin model that corresponds to the defined feature set of the machine learning model underlying the intelligent agent. Since each intelligent agent is trained and/or fine-tuned to perform a particular set of predictive tasks, the features that are utilized by the machine learning models of each intelligent agent will vary. For example, an intelligent agent that is used to diagnose Parkinson's disease will rely on a different set of features than an intelligent agent that simulates the efficacy of a weight-loss drug.

The digital twin is a comprehensive model of the patient and is used to populate the required feature sets for each of the intelligent agents. Each intelligent agent can be mapped to different portions or subsections of the digital twin model, so that the required data for the intelligent agent can be populated from the digital twin model when the intelligent agent is invoked. When an intelligent agent is invoked, the relevant portions of the digital twin can first be identified (i.e., based on the mappings between intelligent agents and the digital twin). After identification, feature extraction is performed and the relevant portions of the digital twin are used to determine feature values for the features in the machine learning model corresponding to the intelligent agent, as well as the particular inferences/tasks being performed. Optionally, feature weighting can also be performed based upon data in the digital twin model, as well the weightings utilized by the intelligent agent for various inferences.

The following table summarizes different ways in which intelligent agents interact with digital agents, along with examples:

| | How the AI Agent Interacts with | |
|---|---|---|
| Function | Digital Twins | Example |
| Analyzing | Identifies patterns, extracts insights, | Early detection of heart failure |
| Complex Datasets | and detects anomalies from EHR, | through biometrics and |
| | wearables, and genomic data. | historical trends. |
| Predictive Risk | Compares digital twin data to vast | Predicting Type 2 diabetes risk |
| Modeling | datasets to predict future health | in a prediabetic patient. |
| | conditions. | |
| Clinical Scenario | Tests different treatment options | Optimizing insulin therapy for |
| Simulation | before applying them to a real patient. | a diabetic patient. |
| Real-Time Decision | Sends AI-generated alerts and | Immediate intervention for a |
| Support | recommended actions based on live | hypertensive crisis. |
| | patient data. | |
| Treatment | Uses simulations to determine which | Selecting the best drug |
| Optimization | medication or therapy is most effective | combination for hypertension |
| | for a patient. | control. |
| Remote | Continuously updates the digital twin, | Notifying a physician when a |
| Monitoring & | alerting caregivers of critical changes. | stroke patient shows worsening |
| Intervention | | symptoms. |

These functions are described in greater detail below, along with examples of different use cases.

At step 105 of FIG. 1, an output is transmitted based at least in part on the generated prediction. The output can take a variety of forms, depending on the specific intelligent agent and the use-case. In the case of diagnosis or triage, the output can be an output in a user interface which displays the predicted diagnosis, along with recommendations for treatment and/or medicines. The output in this scenario can also include a report or summary of the patient's history, symptoms, and predictions regarding diagnosis for a physician or medical professional to review. In the case of treatment simulation, the output can include a summary, report, or chart showing different treatment options, along with predicted outcomes, such as efficacy, side effects, timeline, etc. Many different types of output are contemplated, and these examples are not intended to be limiting. The output can be electronic, in the form of electronic files or an output on a screen, as well as manual, such as a printout of predictions or results.

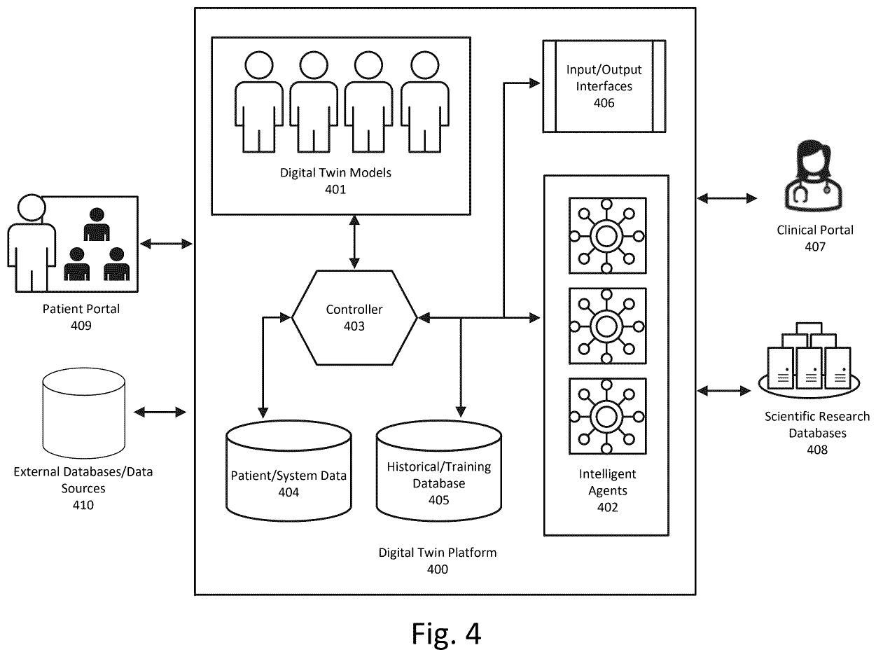

FIG. 4 illustrates a system diagram of a digital twin platform and coupled components according to an exemplary embodiment.

The digital twin platform 400 includes a plurality of digital twin models 401 corresponding to a plurality of patients/users. The platform 400 further includes a plurality of intelligent agents 402, such as the ones discussed above. A historical/training database 405 stores historical data used for training the intelligent agents 402 and can store a variety of information pertinent to different predictive tasks performed by the intelligent agents 402. For example, the historical/training database can store anonymized clinical data, studies, demographic data, drug interaction data, drug efficacy studies, treatment efficacy data and studies, healthcare information and trend data, historical biometric data corresponding to different conditions and diseases, localized data corresponding to particular locales, diagnostic data, mental health data, health risk data, and/or any other data that is utilized for training the intelligent agents.

The platform 400 additionally includes one or more databases storing patient and/or system data 404. The patient data can include additional data about the patient that is not part of the digital twin and/or is not relevant to the healthcare related predictions performed using the digital twin. The patient data can include, for example, current symptoms a patient is experiencing (i.e., input as part of a virtual checkup or triage), patient preferences, communication information (email, address, telephone number, etc.), or other non-health related patient data. The patient data can also include all of the data that is used to generate the digital twin, as discussed earlier. The system data can include data about medical service providers and healthcare providers, as well as any other data used by digital twin platform.

One or more input/output interfaces 406 allow for the exchange of information with the digital twin platform 400. The input/output interfaces can include APIs configured to connect the platform 400 with external databases or repositories, hospital or medical provider systems, insurance systems, websites, or other external systems. Input interfaces can additionally include user interfaces, websites, web portals, mobile apps, or applications that allow patients/users, medical professionals, or others in the healthcare industry to provide inputs to the system. Additionally, output interfaces can include screens, communications interfaces, user interfaces, websites, web portals, mobile apps, or applications that provide output to patients/users, medical professionals, or other users of the system.

A controller 403 on the digital twin platform 400 is configured to control and coordinate the different components on the platform. The controller can serve as an orchestrator and can include instructions for training the various intelligent agents 402 using training data 405, populating or gathering the training data 405 from external sources, such as external databases/data sources 410, performing various routines, selecting the appropriate intelligent agents for various tasks, coordinating interactions between various components (such as the digital twin and the intelligent agents), and generally managing operation of the digital twin platform.

A patient portal 409 can be used by patients to access the digital twin platform 400 from external computing devices. The patient portal 409 can include interfaces and functionality for patients to have a virtual visit, enter symptoms, set up an account and digital twin, run health related assessments and predictions, and perform any of the patient-facing functionality described herein.

A clinical portal 407 can be used by medical professionals to access the digital twin platform. Doctors and nurses ca use the clinical portal to gather information about patients, run simulations of different treatment options, design treatments and treatment plans based on predictions, or additional clinical tasks based on the predictions generated by the system.

As shown in FIG. 4, the digital twin platform 400 can communicate with external databases and data sources 410. These can be databases storing health records used to generate the digital twin, as well as biometric databases storing biometric data corresponding to different user devices or genomic databases storing genetic information corresponding to different users. The external databases can also provide any other health or healthcare related information used for training the intelligent agents or used as part of the inference process, such as disease or drug databases.

The digital twin platform 400 can additionally include an Engine Manager, which is a backend application that exposes the functionalities of creation and management of medical reports, and the management of a conversational experience for the triage process through REST requests. This backend exposes a service that is developed in C# using NetCore 6 technology, employs the ORM Entity Framework to handle database access, control and data migrations. It handles access to resources such as different databases, collects external information stored in Google Healthcare, caches temporary data for rapid consumption, and controls the security internal resources.

A key feature of the backend service is to ensure the integrity and security of the data stored and shared with intelligent agents. To consume this service, authentication is required through an API-KEY, which acts as an additional security mechanism. To ensure optimal performance and scalability, the service incorporates a retry policy for possible failures in the various resources. In cases of sustained failures, a temporary blocking of the resource is implemented to prevent denial of service (DoS) attacks.

This backend service exposes the different functionalities and documentation of how to use it through an API documentation such as Swagger, where it is documented how each endpoint should be consumed and the return codes.

The digital twin platform 400 can additionally be coupled to scientific research databases 408. These databases can store scientific research and the most current findings in different fields. The information retrieved from these databases can be used for further training/refinement of the intelligent agents, to ensure that predictions are in-line with the most current understandings in the health and medical space.

Once the digital twin is constructed, AI-powered intelligent agents interact with it to optimize diagnostics, treatments, and monitoring.
Hospitium.AI's digital twin architecture enables precision medicine, real-time monitoring, and AI-powered diagnostics by:
Integrating structured (EHR/EMR), real-time (wearables), and genomic data.
Utilizing AI to continuously update, analyze, and simulate patient health states.
Providing AI-driven insights and personalized healthcare via intelligent agents.

This approach bridges the gap between fragmented healthcare data and proactive, AI-powered medicine, enabling doctors, hospitals, insurers, and governments to deliver more effective, efficient, and scalable healthcare solutions.

As discussed above, a variety of different healthcare related predictions and tasks can be performed using the present system. Some of these tasks and predictions will now be described in greater detail.

Hospitium.AI is designed to enable intelligent agents to interact seamlessly with digital twins by leveraging machine learning, predictive modeling, and real-time data processing to analyze complex datasets, simulate clinical scenarios, and generate actionable insights.

Hospitium.AI's intelligent agents Gallenus, Séneca, and Flourish-interact seamlessly with the digital twin by leveraging machine learning, predictive modeling, and real-time data processing to analyze complex datasets, simulate clinical scenarios, and generate actionable insights. These interactions are structured through a multi-layered AI architecture that integrates electronic health records (EHRs), biometric data from wearable devices, genomic insights, and behavioral health metrics to create a dynamic, continuously evolving patient model.

The system analyzes complex datasets by collecting and integrating structured and unstructured health data through FHIR and HL7 standards, ensuring interoperability across different healthcare platforms. Natural Language Processing (NLP) extracts insights from physician notes, lab reports, and diagnostic records, while machine learning models analyze historical and real-time data to identify patterns in disease progression, treatment response, and mental health trends. Additionally, AI-driven anomaly detection and risk prediction algorithms detect early warning signs of health deterioration, flagging critical changes in vital signs, mental health markers, and disease risk factors to enhance clinical decision-making.

Hospitium.AI's digital twin enables in silico simulations, allowing clinicians to model disease progression, treatment outcomes, and patient-specific responses in a risk-free virtual environment. This includes testing different therapeutic options, optimizing medication dosages, predicting behavioral responses to mental health interventions, and simulating lifestyle changes. Séneca applies this approach to mental health by modeling patient responses to CBT-based interventions, while Flourish predicts habit formation success rates and engagement levels for well-being interventions. Gallenus, as the core AI-powered intelligent agent, can simulate complications, medication interactions, and treatment adjustments, allowing clinicians to refine personalized treatment strategies dynamically.

The system translates complex data analysis and simulations into actionable insights through dynamic health dashboards, which provide clinicians with real-time patient risk scores, treatment effectiveness reports, and behavioral health analytics. AI-powered decision support suggests personalized therapies, lifestyle modifications, and mental health interventions, while automated alerts notify clinicians when critical health risks or anomalies are detected. These capabilities allow Hospitium.AI to transform healthcare into a proactive, data-driven model, improving diagnostic accuracy, treatment optimization, and patient-centered care.

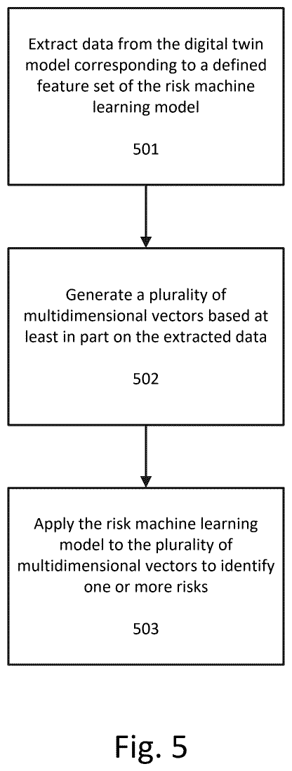

FIG. 5 illustrates a flowchart for applying an intelligent agent in the one or more intelligent agents to the digital twin model based at least in part on extracting data from the digital twin model corresponding to the defined feature set of the machine learning model and applying the machine learning model to the extracted data to generate a prediction when the one or more intelligent agents include a risk prediction intelligent agent according to an exemplary embodiment.

In this case, the one or more intelligent agents comprise a risk prediction intelligent agent comprising a risk machine learning model configured to generate one or more risk predictions. The risk predictions can relate to various different health risks that are identified based at least in part on the digital twin model.

At step 501 data is extracted from the digital twin model corresponding to a defined feature set of the risk machine learning model. The data can be pertinent to the specific type of risk that is being assessed by the risk prediction intelligent agent. Different risk conditions, such as heart disease, cancer, etc., can have different sets of data that are required for the risk assessment.

At step 502 a plurality of multidimensional vectors are generated based at least in part on the extracted data. The multidimensional vectors are also referred to as embeddings, and represent the different feature values in an N-dimensional vector space that is utilized by the machine learning model during inference. The process of embedding projects textual/data values into a high-dimensional latent space, represented as a vector. The vector can have any number of dimensions, such as several hundred dimensions or over a thousand dimensions. The dimensions can represent different features in the feature set. This effectively converts the extracted data into a multidimensional data structure storing numerical values that are utilized by large language models (LLMs) of the intelligent agent.

At step 503 the risk machine learning model is applied to the plurality of multidimensional vectors to identify one or more risks. The identified risks can be those which the intelligent agent deems to meet a predefined threshold probability. Various examples of this risk prediction process and results are explained below.

Hospitium.AI's intelligent agents, including Gallenus, play a central role in analyzing complex datasets, simulating clinical scenarios, and providing actionable insights by continuously interacting with digital twins. These agents integrate real-time patient data, predictive analytics, and machine learning models to support clinical decision-making.

Hospitium.AI's intelligent agents extract, clean, and process large-scale patient data from EHRs, wearables, sensors, and genomic profiles. The agents apply machine learning, natural language processing (NLP), and statistical modeling to identify patterns, detect anomalies, and generate predictions.

### Step-by-Step Example: Identifying Early-Stage Heart Failure Risk

### Step 1: Data Ingestion

The digital twin continuously collects and updates structured and unstructured data.
EHR Data: Blood pressure, cholesterol levels, echocardiogram results.
Wearables & Sensors: Resting heart rate, HRV (heart rate variability), step count, SpO2.
Genomic Data: Polygenic risk score indicating predisposition to cardiovascular diseases.
Clinical Notes: Physician's free-text notes analyzed using NLP.

### Step 2: Feature Engineering & Data Cleaning

The agent normalizes data from different sources to ensure interoperability.
Noise reduction algorithms filter out device errors or inconsistent clinical readings.
The AI model assigns weighted importance to each dataset type.

### Step 3: Pattern Recognition & Predictive Analytics

The intelligent agent and machine learning model compare the patient's digital twin data to a dataset of millions of patients with heart failure.
The AI detects early warning signs, such as:
Decreasing HRV over 3 months.
Frequent shortness of breath detected in wearable oxygen saturation levels.
Elevated NT-proBNP biomarker levels in lab results.

### Step 4: Generating Actionable Insights

The system flags “high risk of early-stage heart failure” and generates a structured report for the clinician.
Recommended actions:
Schedule a cardiology consult.
Adjust blood pressure medication.
Recommend a low-sodium diet and increased physical activity.
The physician receives a summary on their dashboard with AI-validated insights.

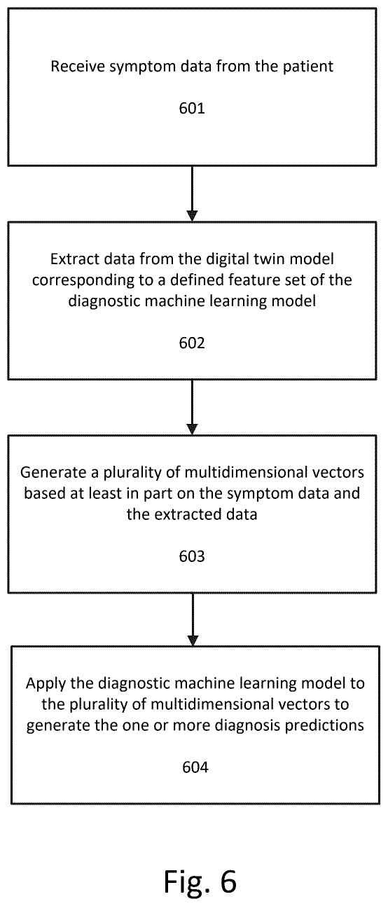

FIG. 6 illustrates a flowchart for applying an intelligent agent in the one or more intelligent agents to the digital twin model based at least in part on extracting data from the digital twin model corresponding to the defined feature set of the machine learning model and applying the machine learning model to the extracted data to generate a prediction when the one or more intelligent agents include a diagnostic intelligent agent according to an exemplary embodiment.

In this case, the one or more intelligent agents comprise a diagnostic intelligent agent comprising a diagnostic machine learning model configured to generate one or more diagnosis predictions.

At step 601 symptom data is received from the patient. This symptom data can be elicited by a chatbot in a user interface as part of an automated triage process.

The chatbot aims to exchange information with the patient to assess the severity of their condition. This classification process is called “triage” and can be standardized according to the Manchester Triage System, used to evaluate the severity of medical emergencies. The chatbot follows a script that asks questions related to the urgency of the situation, in line with the Manchester Triage guidelines. It collects information to assess the emergency status and shares this data with the corresponding care unit, which will make the final evaluation. In case of a maximum urgency situation, control is immediately transferred to a specialized entity.

The patient messages go through safety guard rails. Safety guards are control mechanisms used to ensure that the conversation follows a secure script. This means that both the user and the model must maintain their questions and answers in a way that does not alter the chatbot's script, ensuring the conversation aligns with the Manchester Triage guidelines, using respectful and non-offensive language. To achieve this, an LLM is employed to classify the sentiment and intent of the message. If the message violates any of the established principles, the user is asked to rephrase their message while adhering to the proper language and purpose. If the issue lies within a response generated by the model, it will be regenerated until it meets the required intent, format, and script.

A scenario where intervention may be necessary is when the user asks the chatbot a question that is unrelated to its purpose, such as asking “What is wrong?”. In this case, the chatbot will kindly respond by explaining that it is not designed to answer such questions, clarifying that its role is to assess the user's situation and assist a medical specialist in managing it. For example:
User: Hello, good morning, do you know what's wrong with me?
Chatbot: Good morning, I am not equipped to tell you what is wrong. My role is to gather information about your health status and share it with a specialized entity that will assess you.

The chatbot determines the urgency code using the Manchester Triage System. While the conversation continues, the chatbot generates questions to check the patient's responses. At the start of the conversation, it looks for information in the patient's responses that indicates a high emergency level (red, orange). If the emergency level is not high, it later determines other urgency codes (yellow, green, blue).

The following example illustrates sample messages and a determined urgency code, as shown in the headache conversation below:
User: Hi, I have a headache
Chatbot: Would you describe your headache as mild, moderate, or severe?
User: the pain is severe
Chatbot: On a scale from one to ten, with one being the least pain and ten being the worst pain imaginable, how would you rate your headache?
User: it hurts me a 5
Chatbot: since when have you had this headache?
User: I have been in pain for an hour
Chatbot: Have you taken any medication for your headache?
User: A paracetamol pill
Chatbot: This conversation has concluded. We will now make an appointment for you.
Classification: Yellow Code

At step 602 data is extracted from the digital twin model corresponding to a defined feature set of the diagnostic machine learning model. The specific data that is extracted can depend on the specific intelligent agent and the type of diagnostic assessment that is being performed. Different symptoms, patient history, or other factors can trigger different types of diagnostic assessments.

At step 604 a plurality of multidimensional vectors are generated based at least in part on the symptom data and the extracted data. The process for generating vectors is described above, and this step converts the data corresponding to the relevant features into an N-dimensional vector corresponding to the vector space of the machine learning model.

At step 605 the diagnostic machine learning model is applied to the plurality of multidimensional vectors to generate the one or more diagnosis predictions. The identified diagnosis predictions can be those which the intelligent agent deems to meet a predefined threshold probability. Various examples of this diagnosis prediction process and results are explained further below.

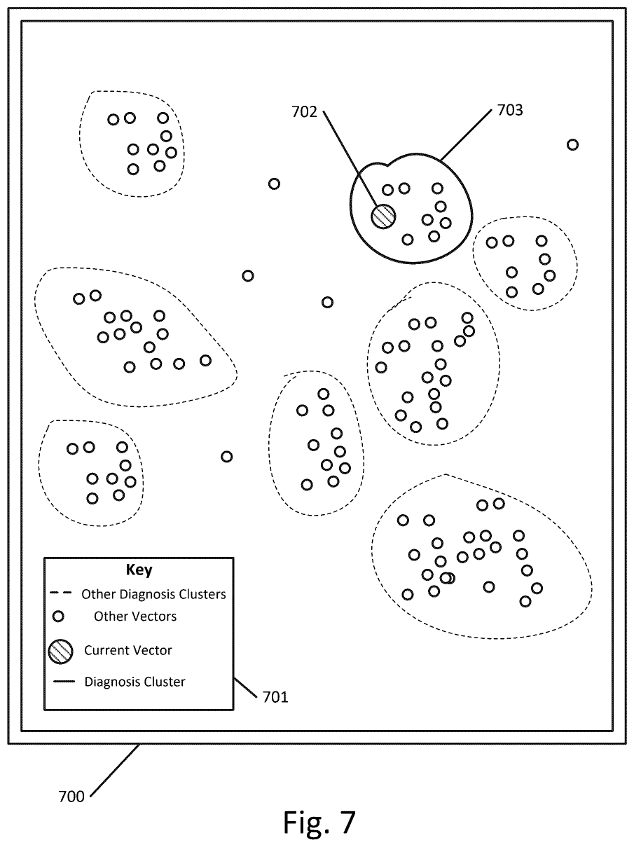

FIG. 7 illustrates an example vector space used to predict diagnosis according to an exemplary embodiment. FIG. 7 shows a two-dimensional vector space, which is presented for ease of illustration only. In operation, the vector space of any particular machine learning model and large language model will have hundreds or thousands of dimensions.

As shown in the key 701, the vector space includes vectors corresponding to different sets of data (i.e., different sets of feature values), represented as white circles. The dashed lines indicate groups of vectors corresponding to certain diagnoses. The diagnoses are grouped into clusters, indicating which sets of data fall within various diagnosis clusters.

A current vector 702 that is being evaluated is shown as a larger circle with a diagonal line pattern. This current vector 702 corresponds to the extracted data/feature values for which a diagnosis is being requested and can include, for example, symptom data and patient data. As shown in FIG. 7, the current vector 702 falls within cluster 703, which is identified as the diagnosis cluster 703. This means that the intelligent agent predicts a diagnosis corresponding to the diagnosis cluster 703 for the current vector 702. For example, if the diagnosis cluster 703 corresponds to lupus and the extracted data/feature values result in the current vector 702, then the intelligent agent can predict that extracted data/feature values indicate a likelihood of a lupus diagnosis. A predetermined distance threshold can be used to determine the cluster sizes. The distance threshold can also be automatically computed based on clustering algorithms that cluster the vectors.

The vector database is used for the process of correlating differential diagnoses. For this purpose, a set of protocols is employed to diagnose high-impact diseases. For each disease, a set of key characteristics is identified and correlated by sections, with each characteristic stored in the database under a specific label. Using information extracted from the patient's medical history and data gathered during the consultation, the information is correlated to identify the most likely diagnoses based on the patient's symptoms.

To perform differential diagnoses, both the patient's medical history data and the information gathered during the medical consultation are used. This data is combined and restructured according to the guidelines and criteria established in the various sections of the diagnostic protocols. Each section is designed to address a specific aspect of the disease or condition being evaluated. Once the data has been processed, the correlation between the collected information and the most relevant protocols is sought. This correlation is carried out through analysis in a vector database, which allows for the identification of patterns and relationships that guide the identification of the most likely diagnoses. This process ensures that the diagnoses are aligned with the most accurate and up-to-date medical standards.

The data is compartmentalized into specific sections according to the protocol of each disease. These sections include information about the target population, clinical manifestations, symptoms of potential complications, and risk factors. The patient and consultation data are restructured and formatted following the structure of each section, enabling efficient preprocessing and retrieval of differential diagnoses.

The system is backed by a medical protocol vector database. The protocols are written and reviewed by a committee of medical personnel responsible for approving each protocol going into the system. Once written, reviewed, and approved, the protocols undergo a chunking and embedding process to produce vectors that contain the semantic meaning of each chunk. Patients' data is parsed and compartmentalize to produce embeddings in a variety of permutations of the different components and to perform a similarity search against the vector database. Each algorithm returns a ranking list of relevant results (e.g., relevant diagnoses). These results undergo a re-ranking process that reduces and eliminates any irrelevant results. The top results of each algorithm are used to produce the inference and the confidence of such based-on representation within each algorithm. The results with the highest confidence level are presented as the differential diagnoses.

A large language model (LLM) is used by the diagnosis intelligent agent to summarize the medical history, assess risk factors, and classify the diagnosis, determining whether it is likely or not, along with providing an explanation. These processes are carried out through a protocol, which is a specialized guide that the model follows to evaluate whether the symptoms match the disease in question.

The diagnosis intelligent agent (which forms part of Gallenus) generates medical inferences from conversations using a structured triage process guided by the Manchester Triage System (MTS). The Triage module is responsible for interacting with the user, ensuring that the conversation follows a predefined medical framework to assess the level of urgency accurately. This approach guarantees consistency, medical validity, and prioritization of critical cases. The process of forming inferences from conversations is described below in greater detail.

The diagnosis intelligent agent/Gallenus follows a four-stage inference process to derive clinically relevant insights from patient interactions:

### Step 1: Structured Triage Process Based on the Manchester Triage System (MTS)

The triage module initiates the conversation using the MTS guideline, which determines the sequence of questions required to assess symptom severity.
The system categorizes the chief complaint (e.g., chest pain, fever, dizziness) into predefined clinical pathways, ensuring that key diagnostic questions are covered.
Based on the patient's responses, the urgency level is assigned according to MTS color codes (Red=Immediate, Orange=Very Urgent, Yellow=Urgent, Green=Standard, Blue=Non-Urgent).

### Step 2: NLP & Named Entity Recognition (NER) for Medical Context Understanding

Natural Language Processing (NLP) models analyze patient input, extracting symptoms, duration, severity, and contextual risk factors.
Named Entity Recognition (NER) identifies specific medical terms, including medications, conditions, recent procedures, and comorbidities.

### Step 3: AI-Driven Clinical Reasoning & Urgency Assessment

The triage model cross-references responses with the MTS framework, ensuring the conversation follows medically validated questioning patterns.
Differential diagnosis models analyze symptom patterns and provide an initial assessment of probable conditions and risk stratification.
The digital twin is consulted to refine the inference based on patient history, previous treatments, and vital records.

### Step 4: Generating the Response & Next Steps

The system provides a triage recommendation, classifying the case as emergency, urgent care, or routine follow-up.
If the situation is high-risk, the system immediately advises medical intervention (e.g., “Call emergency services now”).
For moderate cases, AI-assisted recommendations are provided based on clinical best practices

The following example illustrates the triage process performed by the diagnosis intelligent agent/Gallenus.
Case: A 55-Year-Old Male with Chest Pain
User Input: “I have chest pain and feel short of breath.”
Inference Process:
   MTS triage activation: The system categorizes “chest pain” under Cardiac Evaluation Pathway.
   Structured questioning begins: “When did the pain start? Is it constant or intermittent? Does it spread anywhere?”
   AI cross-references urgency indicators: The patient reports pain radiating to the left arm→System flags as high risk (Red—Immediate).
   Final recommendation: “Your symptoms require immediate medical attention. Call emergency services or go to the nearest ER now.”

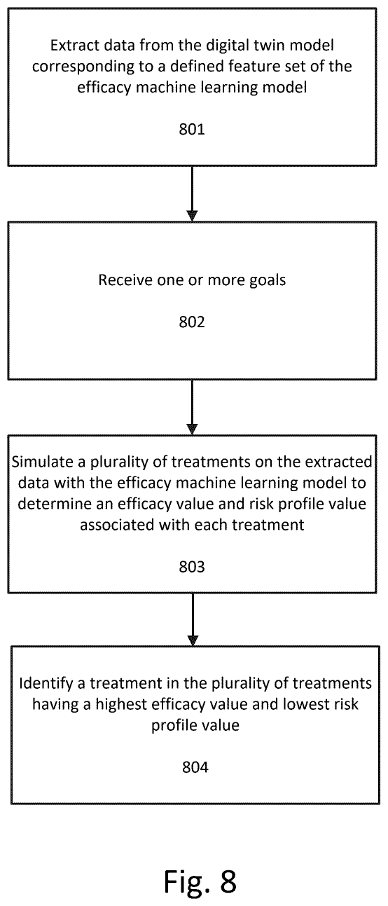

FIG. 8 illustrates a flowchart for applying an intelligent agent in the one or more intelligent agents to the digital twin model based at least in part on extracting data from the digital twin model corresponding to the defined feature set of the machine learning model and applying the machine learning model to the extracted data to generate a prediction when the one or more intelligent agents include an efficacy intelligent agent according to an exemplary embodiment. As discussed previously, the digital twin is a digital replica of the patient, containing the most detailed medical information about the patient, allowing agents to perform simulations with that data.

In this case, the one or more intelligent agents comprise an efficacy intelligent agent comprising a efficacy machine learning model configured to generate one or more efficacy predictions.

At step 801 data is extracted from the digital twin model corresponding to a defined feature set of the efficacy machine learning model. This data can include data models that allow the intelligent agent to model changes in the digital twin based on various inputs/treatments/stimuli.

At step 802 one or more goals are received. The one or more goals specify the desired outcome of the treatment, such as complete abatement of symptoms, a desired timeline, one or more conditions (e.g., a prohibition on certain side effects, a very low percentage chance of serious side effects, etc.), a behavioral change, or any other desired result from a treatment or drug regimen.

At step 803 a plurality of treatments are simulated on the extracted data with the efficacy machine learning model to determine an efficacy value and risk profile value associated with each treatment. The simulation process can include encoding the changes to the extracted data and the digital twin that are caused by each treatment and executing the various treatments to transform the digital twin as indicated by prediction models.

At step 804 a treatment in the plurality of treatments is identified that has the highest efficacy value and/or lowest risk profile value. This step can include comparing the various sets of simulated data to determine which set of data is nearest to the data representing the one or more goals, while filtering out treatments having levels of risk above a predetermined threshold. Examples of the treatment simulation process are described below.

Simulation allows physicians to test different treatment strategies, evaluate disease progression, and assess intervention outcomes in a risk-free environment using digital twins.

### Step-by-Step Example: Optimizing Diabetes Treatment for a High-Risk Patient

### Step 1: Establishing a Baseline Digital Twin

The AI gathers the patient's real-time physiological, clinical, and behavioral data:
EHR: HbA1c levels (9.2%), medication adherence history.
Wearables: Continuous glucose monitor (CGM) shows spikes above 180 mg/dL after meals.
Genomics: Increased risk of insulin resistance due to genetic markers.
Behavioral Factors: Diet logs show high carbohydrate intake.

### Step 2: Defining Clinical Goals

The AI agent identifies the treatment goal: Reduce HbA1c to below 7.0% within 6 months.

### Step 3: Generating Simulation Models

The AI simulates three possible treatment plans:
Option A (Current Regimen, No Change): AI predicts that HbA1c remains high (~8.5%).
Option B (Increase Metformin, Dietary Adjustments): AI predicts HbA1c reduction to 7.5% but identifies a risk of gastrointestinal side effects.
Option C (Add GLP-1 Agonist, Increase Exercise): AI predicts HbA1c drops to 6.8%, with improved weight management.

### Step 4: Selecting & Implementing the Best Option

The AI agent recommends Option C based on the highest efficacy and lowest risk profile.
The physician reviews the AI-generated report, validates the decision, and adjusts the patient's treatment accordingly.

### Step 5: Monitoring & Adjustments

The AI continuously monitors the patient's glucose fluctuations and medication adherence via wearables.
If needed, further adjustments are suggested in real-time.

Hospitium.AI's in silico simulation capability enables clinicians to virtually test treatment plans and interventions using digital twins. The system integrates AI-driven predictive modeling, patient-specific data analytics, and simulation frameworks to provide clinicians with a virtual testing environment for treatment optimization. The process begins with the retrieval of patient-specific data from electronic health records (EHRs), laboratory results, and historical clinical data, which is used to generate a high-fidelity digital twin that continuously updates with new medical inputs. Once the digital twin is created, AI-driven models simulate treatment scenarios by analyzing disease progression, potential side effects, and expected treatment efficacy based on historical data and predictive algorithms. The system allows clinicians to test different therapeutic approaches, such as adjusting medication dosage, introducing new drugs, or modifying therapy protocols, and then compares simulated outcomes to identify the most effective option. AI-driven decision support tools rank treatment alternatives based on success probability, patient-specific risk factors, and prior treatment efficacy, providing clinicians with a detailed report on predicted outcomes, potential risks, and alternative strategies. Once the optimal treatment is identified, it can be applied in real-world clinical settings, with continuous monitoring to refine future interventions.

The system provides a structured and interactive interface that allows clinicians to input and modify treatment plans within the digital twin environment.

Clinicians can enter treatment protocols through a secure, AI-powered clinician dashboard, which integrates with electronic health records (EHRs) and provides real-time access to patient history, lab results, and previous treatment responses. The system supports different input methods, including:
Manual Entry of Treatment Adjustments: Clinicians can directly input new medications, dosages, lifestyle recommendations, or therapy modifications into the digital twin interface.
Predefined Protocol Selection: The system provides evidence-based treatment templates based on clinical guidelines, allowing clinicians to select standardized protocols for common conditions.
AI-Suggested Treatment Modifications: Based on patient-specific data and predictive analytics, Hospitium.AI's intelligent agents generate recommended treatment adjustments that the clinician can review and approve.
Comparative Scenario Testing: Clinicians can select multiple treatment strategies, and the system will run predictive simulations to compare their potential effectiveness.

Once the treatment is entered, the digital twin will simulate physiological responses using machine learning models trained on clinical datasets. The AI system will generate predictive reports on treatment efficacy, potential risks, and alternative recommendations, assisting clinicians in optimizing patient care.

The digital twin in Hospitium.AI simulates the impact of treatments using AI-driven predictive modeling, physiological data integration, and machine learning algorithms. The system creates a dynamic patient-specific virtual model that can simulate disease progression and treatment outcomes before real-world application.

The simulation process works through the following steps:

### 1. Data Integration & Baseline Modeling

The digital twin is constructed using patient-specific data from electronic health records (EHRs), past medical history, lab results, imaging data, and clinician reports.
AI models establish a baseline physiological profile, capturing organ function, metabolic rates, disease state, and previous treatment responses.

### 2. Treatment Input & AI-Based Response Prediction

The clinician enters a proposed treatment plan (e.g., new medication, dosage adjustment, therapy modification) into the system.
Machine learning models trained on large-scale clinical datasets predict how similar patients have responded to the same intervention.
Mathematical models simulate physiological responses by adjusting virtual parameters such as drug absorption rates, metabolic processing, immune response, and potential interactions with existing conditions.

### 3. Scenario-Based Treatment Simulations

The system runs multiple in silico scenarios, modeling short-term and long-term effects of the treatment under different conditions.
AI analyzes variability in patient response, considering factors such as genetics, comorbidities, and disease progression patterns.
The digital twin compares multiple interventions, ranking them based on effectiveness, potential side effects, and risk of adverse reactions.

### 4. Generation of Predictive Reports & Decision Support

AI generates a comprehensive simulation report, outlining expected treatment effectiveness, potential complications, and alternative recommendations.
The system highlights anomalies or contraindications, allowing clinicians to refine the treatment plan before real-world application.
Continuous monitoring and iterative simulations allow real-time adjustments as new clinical data is incorporated.

The system includes disease-specific simulation models, and refined AI-driven decision-making tools to improve treatment planning and clinical outcomes.

The system provides clinicians with AI-driven predictive reports, real-time alerts, and interactive dashboards to facilitate decision-making based on simulation results.

The system includes various methods for communicating results of simulations to clinicians. These are described in greater detail below.

### Dynamic Clinician Dashboard

The clinician can access a secure, AI-powered interface that displays simulation results, risk assessments, and predicted treatment outcomes.
The dashboard features visual data representations, including graphs, trend analyses, and comparative treatment simulations, allowing clinicians to quickly assess potential intervention strategies.
Interactive filters enable clinicians to compare different treatment scenarios, ranking them based on effectiveness, risk factors, and patient-specific considerations.

### AI-Generated Predictive Reports

After running a treatment simulation, Gallenus (Hospitium.AI's medical decision-support agent) will generate an evidence-based report detailing:
Expected treatment effectiveness (e.g., improvement in biomarkers, reduction in symptoms).
Potential risks and contraindications (e.g., drug interactions, likelihood of adverse effects).
Alternative treatment recommendations based on AI analysis of similar patient cases.
These reports are formatted for easy clinician review and can include structured summaries, detailed explanations, and references to relevant clinical guidelines.

### Automated Alerts & Notifications

If a simulation identifies a high-risk scenario (e.g., severe side effects, contraindicated medication interactions, or rapid disease progression), the system can trigger real-time alerts.
Clinicians receive notifications through the dashboard, and potentially via email or secure hospital communication systems, ensuring timely intervention.

### EHR Integration & Clinical Workflow Compatibility

The system is designed to integrate directly with electronic health records (EHRs) so that clinicians can access AI-generated insights within their existing clinical workflow.
Reports and simulation results are automatically appended to the patient's medical file, allowing for seamless documentation and treatment tracking.

The following scenario provides a step-by-step example of each of the above-described steps when using the efficacy intelligent agent (i.e., part of Gallenus) to simulate treatments.

Scenario: A clinician is evaluating treatment options for a patient with congestive heart failure (CHF) and wants to simulate the impact of switching from an ACE inhibitor to an angiotensin receptor-neprilysin inhibitor (ARNI).

Step 1: Clinician Inputs Treatment Plan into Hospitium.AI

The clinician logs into Hospitium.AI's secure dashboard, which integrates with the hospital's electronic health record (EHR) system.

The patient's digital twin is already populated with historical data, including:
Past CHF-related hospitalizations
Ejection fraction trends from echocardiograms
Current medication regimen and dosage
Kidney function (creatinine levels, glomerular filtration rate—GFR)
Blood pressure and electrolyte levels from prior labs

The clinician selects the treatment intervention: switching from an ACE inhibitor to an ARNI and submits the request for simulation.

### Step 2: AI-Driven Simulation of Treatment Impact

Gallenus, the AI-powered virtual doctor, runs predictive simulations on the patient's digital twin, modeling:
Projected improvement in ejection fraction based on response patterns from similar CHF patients.
Potential effects on kidney function, given the patient's current creatinine and GFR levels.
Risk of hyperkalemia (high potassium levels), a common concern when using ARNI medications.
Likelihood of hospitalization reduction over the next 12 months.

### Step 3: Communicating Results to the Clinician

### Clinician Dashboard Overview

The clinician receives a summary of the simulation results via the Hospitium.AI dashboard:

### Effectiveness Prediction:

Ejection Fraction Improvement: +5% over six months (high probability).
Reduced CHF-related hospitalizations: 25% lower risk based on comparative modeling.

### Risk Assessment:

Renal function impact: Mild decrease in GFR (~5 mL/min)—flagged as “Monitor Closely.”
Hyperkalemia Risk: 10% likelihood of potassium elevation requiring monitoring.

### Alternative Recommendations:

AI suggests starting at a lower ARNI dose to mitigate hyperkalemia risk.
If renal function declines further, consider a SGLT2 inhibitor as adjunct therapy.

### AI-Generated Predictive Report

A detailed PDF report is auto-generated and attached to the patient's EHR, allowing the clinician to review it at any time.

The report includes:
Graphs showing projected kidney function changes over time.
Comparative analysis of CHF-related readmission rates with and without ARNI therapy.

Risk-benefit assessment with AI-generated confidence scores.

### Step 4: Clinician Decision & Real-World Implementation

The clinician reviews the AI-generated insights and decides to:
Proceed with ARNI therapy but at a reduced initial dose.
Schedule lab tests in 4 weeks to monitor potassium and renal function.
Set an automatic AI re-evaluation in 3 months based on updated lab and clinical data.

Hospitium.AI updates the digital twin continuously, tracking real-world treatment response and adjusting predictive models accordingly.

### Step 5: Ongoing Monitoring & AI Alerts

If the patient's potassium levels exceed safe thresholds, Hospitium.AI triggers an alert recommending intervention.
If kidney function worsens beyond AI-predicted parameters, the clinician receives a notification with revised treatment recommendations.

### Auditing and Course Correction of Machine Learning (ML) Inferences in Hospitium.AI

Hospitium.AI integrates a continuous auditing and feedback loop for its AI models, ensuring that inferences remain clinically accurate, ethically sound, and adaptable to real-world patient care. The medical team plays a crucial role in auditing and refining the model's decisions through structured feedback mechanisms.

### 1. Auditing Process for ML Inferences

Hospitium.AI implements three layers of auditing to validate and improve the AI models:

### A. Internal AI Model Validation (Pre-Deployment Phase)

Before any inference is made on live patients, models are pre-trained and validated using:
Retrospective Analysis of Historical Patient Data: AI predictions are compared with actual medical outcomes from anonymized datasets.
Synthetic Data Testing: Digital twins with simulated diseases are used to test AI inference consistency.
Clinical Guidelines Cross-Validation: AI-generated recommendations are benchmarked against evidence-based clinical guidelines (e.g., WHO, AHA, ADA).

### B. Real-Time Auditing During Deployment

When AI inferences are made in live clinical environments, the system has automated safeguards and human oversight:

### 1) Confidence Scoring & Anomaly Detection

Each AI inference generates a confidence score (e.g., 85% certainty that the patient has early-stage diabetes).
If the confidence level is below a pre-set threshold, the AI:
   Flags the case for human review.
   Recommends alternative diagnoses or treatment options for physician comparison.
   Anomaly Detection Algorithms: Identify outliers or inconsistencies that differ significantly from standard medical knowledge.

### 2) Shadow Mode Learning & Parallel Decision Review

AI models run alongside human decision-making but do not automatically apply recommendations.
The medical team reviews AI-generated reports, comparing them against clinical decisions before full automation is allowed.

### 3) Post-Treatment Outcome Monitoring

After a clinician follows a treatment suggested by the AI, the system monitors real-world outcomes.
Discrepancies between expected and actual results are flagged for further evaluation.

### 2. Medical Team Feedback and Model Corrections

Hospitium.AI enables continuous model improvement through human-in-the-loop (HITL) mechanisms. Clinicians and auditors directly influence AI learning through structured feedback loops:

### A. Manual Physician Feedback Via the Hospitium.AI Dashboard

Physicians can approve, modify, or reject AI-generated recommendations.
Structured Feedback Form:
   Was the AI recommendation correct? (Yes/No)
   If incorrect, what was the correct diagnosis/treatment?
   Severity of mistake (Minor/Major/Critical)
      B. Reinforcement Learning from Human Feedback (RLHF)
Corrected cases are fed back into the model, retraining the AI using supervised learning techniques.
Over time, the AI adapts to region-specific clinical variations, improving predictive accuracy.

### C. Model Retraining & Deployment Cycles

AI models are updated every 3-6 months, integrating the latest clinical feedback and research.
New versions are tested in controlled environments before full release.

### 3. Regional and Global Auditing Teams

The medical auditing structure is designed to ensure localized clinical relevance while maintaining global quality standards:

### A. Regional Medical Oversight

Each geographic region has a localized team of clinicians and auditors to validate AI recommendations.
This ensures that regional variations in disease prevalence, medical guidelines, and cultural factors are accounted for.

### B. Central AI Governance Team

A global AI oversight committee ensures compliance with international healthcare standards (e.g., HIPAA, GDPR, ISO 13485 for AI in healthcare).
Ensures that AI updates are aligned with the latest global medical research and regulatory policies.

### 4. Example of AI Auditing in Action: Detecting Errors in Diagnosing Pneumonia

### Step 1: AI Model Generates an Inference

AI detects pneumonia probability: 88% confidence based on:
Chest X-ray image.
Patient's vitals (elevated temperature, rapid breathing).
Prior history of respiratory infections.

### Step 2: Physician Review & Feedback

Doctor reviews AI suggestion but notes that symptoms better match tuberculosis (TB).
Doctor overrides AI recommendation, labels the case as TB, not pneumonia.

### Step 3: AI Model Adjusts via Reinforcement Learning

AI updates its weightings, giving more importance to risk factors for TB in similar future cases.
Future cases with similar patterns will now prioritize TB as a differential diagnosis.

### Step 4: Quality Control & Dashboard Updates

AI tracks all overridden recommendations and reports them in a weekly auditing dashboard.
If AI accuracy for pneumonia falls below 85%, the system initiates immediate retraining.

Hospitium.AI integrates real-time physician oversight, human feedback, and continuous model retraining to ensure that AI-generated recommendations remain:
Accurate and aligned with clinical best practices.
Adaptable to regional and hospital-specific guidelines.
Fully auditable, traceable, and compliant with regulatory standards.

This AI governance framework ensures that Hospitium.AI delivers safe, effective, and continuously improving healthcare solutions worldwide.

Unlike many AI systems that operate as black-box models with limited transparency, Hospitium.AI is 100% auditable, meaning that every inference generated by the system can be traced, reviewed, and validated at any point. The auditing strategy (100%, 75%, 50%, 25%) refers to the progressive levels of human oversight in model validation, but regardless of the percentage of active review, every decision made by the AI remains fully auditable.

Different versions of machine learning models can be used for the intelligent agents that form part of the platform. Specifically, master versions of the machine learning models can serve as foundational models, and local instances of each machine learning model can be instantiated for different localities. This process is described in greater detail below.

The intelligent agents operate using a centralized master ML model that serves as the foundation for all locality-specific instances. Instead of building each local version from scratch, the system follows a two-stage training process:

Global Pretraining on a Master Model—The core ML model is trained on a large, diverse dataset consisting of international clinical guidelines, global patient case studies, and broad-spectrum medical literature.

Local Fine-Tuning & Adaptation—Each deployment is further trained using regional clinical data, local healthcare policies, and population-specific trends to customize recommendations for specific healthcare environments.

### 1. Training Process for Customizing Each Local Instance

### Step 1: Global Pretraining on a Master Model

The base model is trained on a large-scale, multi-region medical dataset, incorporating:
International clinical guidelines (WHO, NIH, FDA, EMA).
Diverse patient case studies across different demographics.
Medical ontologies and knowledge graphs (ICD-10, SNOMED CT, UMLS).
General disease progression models based on population-wide epidemiological data.

The result is a generalized AI system capable of handling broad clinical reasoning across multiple specialties.

### Step 2: Local Adaptation Using Region-Specific Data

Once the master model is deployed in a new locality, it undergoes local fine-tuning through a supervised learning process:

### Integration of Local Clinical Guidelines

The AI is retrained using region-specific healthcare protocols (e.g., NICE guidelines in the UK, CDC protocols in the US, and country-specific drug formularies).
Customization includes medication availability, diagnostic preferences, and treatment workflows unique to the region.

### Adaptation to Population-Specific Health Trends

The system incorporates local epidemiological data, ensuring accurate risk stratification based on prevalent conditions (e.g., high diabetes prevalence in the Middle East, increased cardiovascular risks in the US).
AI models adjust for genetic, environmental, and socioeconomic factors that influence disease patterns.

### Training on Local Language & Cultural Variations

The NLP component of Gallenus is fine-tuned to understand local dialects, medical terminology, and cultural communication nuances.
AI adjusts for language-specific symptom descriptions (e.g., how patients in different cultures describe pain or discomfort).

### Step 3: Continuous Learning & Region-Specific Model Updates

The system continuously refines itself through:
Real-world clinician feedback loops that update the AI's diagnostic accuracy.
Local auditing teams that monitor AI recommendations against regional clinical best practices.
Regular model retraining using anonymized, de-identified patient cases from local healthcare institutions.

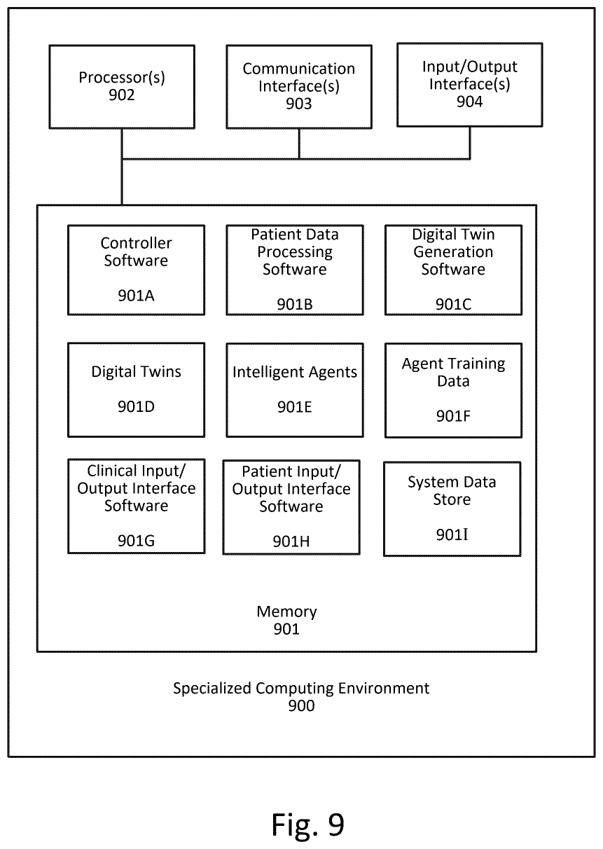

The above-described techniques are implemented using one or more special-purpose computer systems having computer-readable instructions loaded thereon that enable the computer system to implement the above-described techniques. FIG. 9 illustrates a specialized computing environment 900 for generating predictions with a digital twin architecture according to an exemplary embodiment.

With reference to FIG. 9, the computing environment 900 includes at least one processing unit/controller 902 and memory 901. The processing unit 902 executes computer-executable instructions and can be a real or a virtual processor. In a multi-processing system, multiple processing units execute computer-executable instructions to increase processing power. The memory 901 can be volatile memory (e.g., registers, cache, RAM), non-volatile memory (e.g., ROM, EEPROM, flash memory, etc.), or some combination of the two. The memory 901 can store software and data used for implementing the above-described techniques, including controller software 901A, patient data processing software 901B, digital twin generation software 901C, digital twins 901D, intelligent agents 901E, agent training data 901F, clinical input/output interface software 901G, and patient input/output interface software 901H, as well as a system data store 901I. The memory can store additional software and data, such as data integration software, personalized medicine software, continuous monitoring software, treatment adherence monitoring software, artificial intelligence engines, user interface software, chatbot software, and a patient database.

All of the software stored within memory 901 can be stored as a computer-readable instructions, that when executed by one or more processors 902, cause the processors to perform the functionality described above.

Processor(s) 902 execute computer-executable instructions and can be a real or virtual processors. In a multi-processing system, multiple processors or multicore processors can be used to execute computer-executable instructions to increase processing power and/or to execute certain software in parallel.

Specialized computing environment 900 additionally includes a communication interface 903, such as a network interface, which is used to communicate with devices, applications, or processes on a computer network or computing system, collect data from devices on a network, and implement encryption/decryption actions on network communications within the computer network or on data stored in databases of the computer network. The communication interface conveys information such as computer-executable instructions, audio or video information, or other data in a modulated data signal. A modulated data signal is a signal that has one or more of its characteristics set or changed in such a manner as to encode information in the signal. By way of example, and not limitation, communication media include wired or wireless techniques implemented with an electrical, optical, RF, infrared, acoustic, or other carrier.

Specialized computing environment 900 further includes input and output interfaces 904 that allow users (such as system administrators) to provide input to the system to set parameters, to edit data stored in memory 901, or to perform other administrative functions.

An interconnection mechanism (shown as a solid line in FIG. 9), such as a bus, controller, or network interconnects the components of the specialized computing environment 900.

Input and output interfaces 904 can be coupled to input and output devices. For example, Universal Serial Bus (USB) ports can allow for the connection of a keyboard, mouse, pen, trackball, touch screen, or game controller, a voice input device, a scanning device, a digital camera, remote control, or another device that provides input to the specialized computing environment 900.

Specialized computing environment 900 can additionally utilize a removable or non-removable storage, such as magnetic disks, magnetic tapes or cassettes, CD-ROMs, CD-RWs, DVDs, USB drives, or any other medium which can be used to store information and which can be accessed within the specialized computing environment 900.

Having described and illustrated the principles of our invention with reference to the described embodiment, it will be recognized that the described embodiment can be modified in arrangement and detail without departing from such principles. Elements of the described embodiment shown in software can be implemented in hardware and vice versa.

In view of the many possible embodiments to which the principles of our invention can be applied, we claim as our invention all such embodiments as can come within the scope and spirit of the following claims and equivalents thereto.

1. A method executed by one or more computing devices of a controller for generating predictions with a digital twin architecture, the method comprising:
extracting, by the controller, a plurality of data sets corresponding to a user from a plurality of data sources;
generating, by the controller, a digital twin model corresponding to the user based at least in part on the plurality of data sets;
storing, by the controller, one or more intelligent agents configured to interface with the digital twin model, each intelligent agent comprising a machine learning model corresponding to a defined feature set and being trained on historical data, the machine learning model being configured to generate a prediction, wherein the prediction comprises one or more of: a condition prediction, a risk prediction, or an efficacy prediction;
applying, by the controller, an intelligent agent in the one or more intelligent agents to the digital twin model based at least in part on extracting data from the digital twin model corresponding to the defined feature set of the machine learning model and applying the machine learning model to the extracted data to generate a prediction; and
transmitting, by the controller, an output based at least in part on the generated prediction.

2. The method of claim 1, wherein the plurality of data sources comprise one or more of: clinical records, biometric data, or genetic data.

3. The method of claim 1, wherein generating a digital twin model corresponding to the patient based at least in part on the plurality of data sets comprises one or more of:
natural language processing of unstructured data in the plurality of data sets;
standardization of data formats in the plurality of data sets;
normalization of data in the plurality of data sets;
alignment of data in the plurality of data sets;
error correction of data in the plurality of data sets; or
modeling of one or more relationships between data in the plurality of data sets.

4. The method of claim 1, wherein the one or more intelligent agents comprise a risk prediction intelligent agent comprising a risk machine learning model configured to generate one or more risk predictions and wherein applying an intelligent agent in the one or more intelligent agents to the digital twin model based at least in part on extracting data from the digital twin model corresponding to the defined feature set of the machine learning model and applying the machine learning model to the extracted data to generate a prediction comprises:
extracting data from the digital twin model corresponding to a defined feature set of the risk machine learning model;
generating a plurality of multidimensional vectors based at least in part on the extracted data; and
applying the risk machine learning model to the plurality of multidimensional vectors to identify one or more risks.

5. The method of claim 1, wherein the one or more intelligent agents comprise a diagnostic intelligent agent comprising a diagnostic machine learning model configured to generate one or more diagnosis predictions and wherein applying an intelligent agent in the one or more intelligent agents to the digital twin model based at least in part on extracting data from the digital twin model corresponding to the defined feature set of the machine learning model and applying the machine learning model to the extracted data to generate a prediction comprises:
receiving symptom data from the patient;
extracting data from the digital twin model corresponding to a defined feature set of the diagnostic machine learning model;
generating a plurality of multidimensional vectors based at least in part on the symptom data and the extracted data; and
applying the diagnostic machine learning model to the plurality of multidimensional vectors to generate the one or more diagnosis predictions.

6. The method of claim 1, wherein the one or more intelligent agents comprise an efficacy intelligent agent comprising a efficacy machine learning model configured to generate one or more efficacy predictions and wherein applying an intelligent agent in the one or more intelligent agents to the digital twin model based at least in part on extracting data from the digital twin model corresponding to the defined feature set of the machine learning model and applying the machine learning model to the extracted data to generate a prediction comprises:
extracting data from the digital twin model corresponding to a defined feature set of the efficacy machine learning model;
receiving one or more goals;
simulating a plurality of treatments on the extracted data with the efficacy machine learning model to determine an efficacy value and risk profile value associated with each treatment; and
identifying a treatment in the plurality of treatments having a highest efficacy value and lowest risk profile value.

7. The method of claim 1, wherein the one or more intelligent agents comprise a plurality of intelligent agents and wherein the plurality of intelligent agents comprise one or more of:
risk prediction intelligent agent;
a diagnostic intelligent agent;
an efficacy intelligent agent;
a well-being optimization intelligent agent; or
a mental health support intelligent agent.

8. An apparatus for generating predictions with a digital twin architecture, the apparatus comprising:
one or more processors; and
one or more memories operatively coupled to at least one of the one or more processors and having instructions stored thereon that, when executed by at least one of the one or more processors, cause at least one of the one or more processors to:
extract a plurality of data sets corresponding to a user from a plurality of data sources;
generate a digital twin model corresponding to the user based at least in part on the plurality of data sets;
store one or more intelligent agents configured to interface with the digital twin model, each intelligent agent comprising a machine learning model corresponding to a defined feature set and being trained on historical data, the machine learning model being configured to generate a prediction, wherein the prediction comprises one or more of: a condition prediction, a risk prediction, or an efficacy prediction;
apply an intelligent agent in the one or more intelligent agents to the digital twin model based at least in part on extracting data from the digital twin model corresponding to the defined feature set of the machine learning model and applying the machine learning model to the extracted data to generate a prediction; and
transmit an output based at least in part on the generated prediction.

9. The apparatus of claim 8, wherein the plurality of data sources comprise one or more of: clinical records, biometric data, or genetic data.

10. The apparatus of claim 8, wherein the instructions that, when executed by at least one of the one or more processors, cause at least one of the one or more processors to generate a digital twin model corresponding to the patient based at least in part on the plurality of data sets further cause at least one of the one or more processors to perform one or more of:
natural language processing of unstructured data in the plurality of data sets;
standardization of data formats in the plurality of data sets;
normalization of data in the plurality of data sets;
alignment of data in the plurality of data sets;
error correction of data in the plurality of data sets; or
modeling of one or more relationships between data in the plurality of data sets.

11. The apparatus of claim 8, wherein the one or more intelligent agents comprise a risk prediction intelligent agent comprising a risk machine learning model configured to generate one or more risk predictions and wherein the instructions that, when executed by at least one of the one or more processors, cause at least one of the one or more processors to apply an intelligent agent in the one or more intelligent agents to the digital twin model based at least in part on extracting data from the digital twin model corresponding to the defined feature set of the machine learning model and applying the machine learning model to the extracted data to generate a prediction further cause at least one of the one or more processors to:
extract data from the digital twin model corresponding to a defined feature set of the risk machine learning model;
generate a plurality of multidimensional vectors based at least in part on the extracted data; and
apply the risk machine learning model to the plurality of multidimensional vectors to identify one or more risks.

12. The apparatus of claim 8, wherein the one or more intelligent agents comprise a diagnostic intelligent agent comprising a diagnostic machine learning model configured to generate one or more diagnosis predictions and wherein the instructions that, when executed by at least one of the one or more processors, cause at least one of the one or more processors to apply an intelligent agent in the one or more intelligent agents to the digital twin model based at least in part on extracting data from the digital twin model corresponding to the defined feature set of the machine learning model and applying the machine learning model to the extracted data to generate a prediction further cause at least one of the one or more processors to:
receive symptom data from the patient;
extract data from the digital twin model corresponding to a defined feature set of the diagnostic machine learning model;
generate a plurality of multidimensional vectors based at least in part on the symptom data and the extracted data; and
apply the diagnostic machine learning model to the plurality of multidimensional vectors to generate the one or more diagnosis predictions.

13. The apparatus of claim 8, wherein the one or more intelligent agents comprise an efficacy intelligent agent comprising a efficacy machine learning model configured to generate one or more efficacy predictions and wherein the instructions that, when executed by at least one of the one or more processors, cause at least one of the one or more processors to apply an intelligent agent in the one or more intelligent agents to the digital twin model based at least in part on extracting data from the digital twin model corresponding to the defined feature set of the machine learning model and applying the machine learning model to the extracted data to generate a prediction further cause at least one of the one or more processors to:
extract data from the digital twin model corresponding to a defined feature set of the efficacy machine learning model;
receive one or more goals;
simulate a plurality of treatments on the extracted data with the efficacy machine learning model to determine an efficacy value and risk profile value associated with each treatment; and
identify a treatment in the plurality of treatments having a highest efficacy value and lowest risk profile value.

14. The apparatus of claim 8, wherein the one or more intelligent agents comprise a plurality of intelligent agents and wherein the plurality of intelligent agents comprise one or more of:
risk prediction intelligent agent;
a diagnostic intelligent agent;
an efficacy intelligent agent;
a well-being optimization intelligent agent; or
a mental health support intelligent agent.

15. At least one non-transitory computer-readable medium storing computer-readable instructions for generating predictions with a digital twin architecture that, when executed by one or more computing devices, cause at least one of the one or more computing devices to:
extract a plurality of data sets corresponding to a user from a plurality of data sources;
generate a digital twin model corresponding to the user based at least in part on the plurality of data sets;
store one or more intelligent agents configured to interface with the digital twin model, each intelligent agent comprising a machine learning model corresponding to a defined feature set and being trained on historical data, the machine learning model being configured to generate a prediction, wherein the prediction comprises one or more of: a condition prediction, a risk prediction, or an efficacy prediction;
apply an intelligent agent in the one or more intelligent agents to the digital twin model based at least in part on extracting data from the digital twin model corresponding to the defined feature set of the machine learning model and applying the machine learning model to the extracted data to generate a prediction; and
transmit an output based at least in part on the generated prediction.

16. The at least one non-transitory computer-readable medium of claim 15, wherein the plurality of data sources comprise one or more of: clinical records, biometric data, or genetic data.

17. The at least one non-transitory computer-readable medium of claim 15, wherein the instructions that, when executed by at least one of the one or more computing devices, cause at least one of the one or more computing devices to generate a digital twin model corresponding to the patient based at least in part on the plurality of data sets further cause at least one of the one or more computing devices to perform one or more of:
natural language processing of unstructured data in the plurality of data sets;
standardization of data formats in the plurality of data sets;
normalization of data in the plurality of data sets;
alignment of data in the plurality of data sets;
error correction of data in the plurality of data sets; or
modeling of one or more relationships between data in the plurality of data sets.

18. The at least one non-transitory computer-readable medium of claim 15, wherein the one or more intelligent agents comprise a risk prediction intelligent agent comprising a risk machine learning model configured to generate one or more risk predictions and wherein the instructions that, when executed by at least one of the one or more computing devices, cause at least one of the one or more computing devices to apply an intelligent agent in the one or more intelligent agents to the digital twin model based at least in part on extracting data from the digital twin model corresponding to the defined feature set of the machine learning model and applying the machine learning model to the extracted data to generate a prediction further cause at least one of the one or more computing devices to:
extract data from the digital twin model corresponding to a defined feature set of the risk machine learning model;
generate a plurality of multidimensional vectors based at least in part on the extracted data; and
apply the risk machine learning model to the plurality of multidimensional vectors to identify one or more risks.

19. The at least one non-transitory computer-readable medium of claim 15, wherein the one or more intelligent agents comprise a diagnostic intelligent agent comprising a diagnostic machine learning model configured to generate one or more diagnosis predictions and wherein the instructions that, when executed by at least one of the one or more computing devices, cause at least one of the one or more computing devices to apply an intelligent agent in the one or more intelligent agents to the digital twin model based at least in part on extracting data from the digital twin model corresponding to the defined feature set of the machine learning model and applying the machine learning model to the extracted data to generate a prediction further cause at least one of the one or more computing devices to:
receive symptom data from the patient;
extract data from the digital twin model corresponding to a defined feature set of the diagnostic machine learning model;
generate a plurality of multidimensional vectors based at least in part on the symptom data and the extracted data; and
apply the diagnostic machine learning model to the plurality of multidimensional vectors to generate the one or more diagnosis predictions.

20. The at least one non-transitory computer-readable medium of claim 15, wherein the one or more intelligent agents comprise an efficacy intelligent agent comprising a efficacy machine learning model configured to generate one or more efficacy predictions and wherein the instructions that, when executed by at least one of the one or more computing devices, cause at least one of the one or more computing devices to apply an intelligent agent in the one or more intelligent agents to the digital twin model based at least in part on extracting data from the digital twin model corresponding to the defined feature set of the machine learning model and applying the machine learning model to the extracted data to generate a prediction further cause at least one of the one or more computing devices to:
extract data from the digital twin model corresponding to a defined feature set of the efficacy machine learning model;
receive one or more goals;
simulate a plurality of treatments on the extracted data with the efficacy machine learning model to determine an efficacy value and risk profile value associated with each treatment; and
identify a treatment in the plurality of treatments having a highest efficacy value and lowest risk profile value.

21. The at least one non-transitory computer-readable medium of claim 15, wherein the one or more intelligent agents comprise a plurality of intelligent agents and wherein the plurality of intelligent agents comprise one or more of:
risk prediction intelligent agent;
a diagnostic intelligent agent;
an efficacy intelligent agent;
a well-being optimization intelligent agent; or
a mental health support intelligent agent.
